(19) United States
(12) Patent Application Publication
(10) Pub. No.: US 2021/0267502 A1
(43) Pub. Date: Sep. 2, 2021

(54) WEARABLE DEVICE FOR DETECTING SALINITY IN PERSPIRATION

(71) Applicants: Alex GURSKI, Vienna, VA (US); Thaissa PEIXOTO, Vienna, VA (US)

(72) Inventors: Alex GURSKI, Vienna, VA (US); Thaissa PEIXOTO, Vienna, VA (US)

(21) Appl. No.: 16/987,649

(22) Filed: Aug. 7, 2020

Related U.S. Application Data
(60) Provisional application No. 62/983,051, filed on Feb. 28, 2020.

(51) Int. Cl. A61B 5/145; A61B 5/00; G16H 50/30

(52) U.S. Cl. CPC A61B 5/14517; G16H 50/30; A61B 5/6831; A61B 5/4266

(57) ABSTRACT

A wearable device for determining risk of an underlying health concern is disclosed. The wearable device comprises a replaceable medium for absorbing perspiration of a user, wherein the replaceable medium is at least occasionally in contact with the user. The device also includes a power source electrically connected to the medium, and a resistance detector processor for periodically detecting the electrical resistance of the absorbed perspiration, wherein the electrical resistance of the absorbed perspiration is dependent on the salinity of the perspiration. Further, the device includes a risk detector processor for: (1) determining whether the detected resistance meets at least one condition, and (2) triggering an alert signal if the at least one condition is met, a storage device for storing resistance values; and a warning signal generator for providing a warning signal to the user based on the alert signal.

### CROSS REFERENCE TO RELATED APPLICATIONS

The application claims priority to U.S. Provisional Application 62/983,051, filed Feb. 28, 2020, the contents of which are incorporated herein in its entirety.

### FIELD OF THE INVENTION

This invention relates generally to systems and methods for detecting and measuring salinity in perspiration, and more particularly to wearable systems and methods for detecting and measuring salinity in perspiration to identify the presence or risk of underlying health concerns.

### BACKGROUND OF THE INVENTION

The human body constantly generates signals that can be being harnessed and analyzed to determine an individual's underlying health or genetic characteristics. For example, medical professionals routinely measure a patient's vital signs (e.g., heart rate, pulse, temperature, blood pressure) to identify potential ailments or other health-related concerns. Blood samples may also drawn, for example, to identify the presence of any infections, diseases, or an individual's DNA or other genetic markers.

While not commonly known, perspiration (or sweat) may also useful to identify the presence or risk of certain ailments. For example, the salinity of perspiration can be measured to determine whether an individual is at risk of hyponatremia—a condition that occurs when the level of sodium in the blood is too low. The salinity of perspiration can also signal the presence or risk of cystic fibrosis—a progressive, genetic disease that causes persistent lung infections and limits the ability to breathe over time.

Technologies exist for measuring the salinity of perspiration. For example, European Patent EP0421625B1 discloses a device for continuously measuring the skin local sweating rate including rate of emotional perspiration as well as that of heat-sensitive perspiration. U.S. Pat. No. 6,198,953 discloses a method and system for continuously obtaining and analyzing, on a real time basis, sweat from a selected area of skin on the body of a person. U.S. Pat. No. 8,565,850 discloses a kit for measuring sweat activity. Japanese patent JP4923038B2 discloses the use of textile-based electrodes including a fabric portion having stretch-recovery non-conductive yarns and an electrically conductive region having stretch-recovery electrically conductive yarn filaments.

While the above references disclose systems and methods capable of capturing and/or analyzing perspiration, none describe a wearable system that utilizes algorithms to inform a user of a potential health condition, or that alerts the user of the need to replace a medium for absorbing perspiration. Moreover, none of the above references disclose systems and methods that supplement the collection and analysis of perspiration with the analysis of other biometric signals to determine the presence or risk of underlying health concerns.

These and other problems exist.

### SUMMARY OF THE INVENTION

An object of the present invention is to overcome the aforementioned and other drawbacks existing in the prior art.

The systems and methods described herein measure the salinity of perspiration to determine whether there is a presence or risk of an underlying health concern. Specifically, the systems and methods determine the electrical resistance of perspiration of a user to determine whether certain thresholds are exceeded, and, if so, alert the user to the potential health concern. In some embodiments, the systems and methods describe herein may alert the user to the need to replace an absorbing medium. In some embodiments, the systems and methods described herein may supplement the collection and analysis of perspiration with the analysis of other biometric signals to determine the presence or risk of underlying health concerns.

According to an embodiment of the systems and methods described herein, a wearable device for determining risk of an underlying health concern is provided. The wearable device comprising: a replaceable medium for absorbing perspiration of a user, wherein the replaceable medium is at least occasionally in contact with the user; a power source electrically connected to the medium; a resistance detector processor for periodically detecting the electrical resistance of the absorbed perspiration, wherein the electrical resistance of the absorbed perspiration is dependent on the salinity of the perspiration; a risk detector processor for: (1) determining whether the detected resistance meets at least one condition, and (2) triggering an alert signal if the at least one condition is met; a storage device for storing detected resistance values; and a warning signal generator for providing a warning signal to the user based on the alert signal.

The accompanying drawings, which are incorporated in and constitute a part of this specification, illustrate various embodiments of the invention and, together with the description, serve to explain the principles of the invention.

### BRIEF DESCRIPTION OF THE DRAWINGS

FIG. 1A is an overview of the components of a wearable device for determining risk of an underlying health concern, according to an embodiment of the systems and methods described herein.

FIG. 1B is an overview of the electrical principles and thresholds utilized by the wearable device for determining risk of an underlying health concern, according to an embodiment of the systems and methods described herein.

FIG. 1C is a chart showing the relationship between electrical resistance and salinity of a solution, according to an embodiment of the systems and methods described herein.

FIG. 2 is a method for powering up and detecting the electrical resistance of a new absorption medium of a wearable device for determining risk of an underlying health concern, according to an embodiment of the systems and methods described herein.

FIG. 3 is a method for powering up and detecting the electrical resistance of a used absorption medium of a wearable device for determining risk of an underlying health concern, according to an embodiment of the systems and methods described herein.

FIG. 4A is a method for general operation of a wearable device for determining risk of an underlying health concern, according to an embodiment of the systems and methods described herein.

FIG. 4B is an overview of the components of a wearable device for determining risk of an underlying health concern, according to an embodiment of the systems and methods described herein.

FIG. 5 is an overview of possible implementations of a wearable device for determining risk of an underlying health concern, according to various embodiments of the systems and methods described herein.

### DETAILED DESCRIPTION OF THE INVENTION

Reference will now be made to the present preferred embodiments of the invention, examples of which are illustrated in the accompanying drawings in which like reference characters refer to corresponding elements.

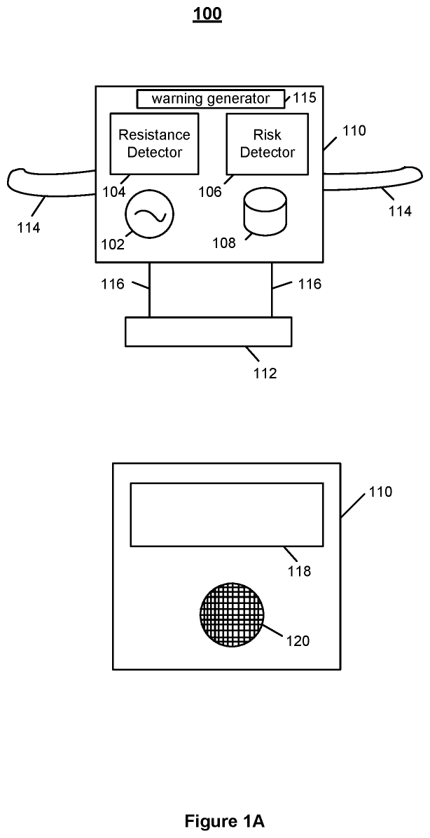

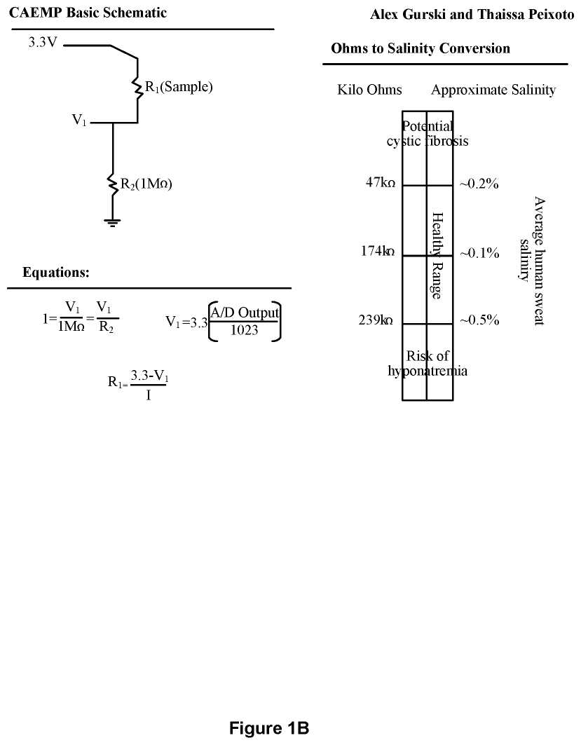

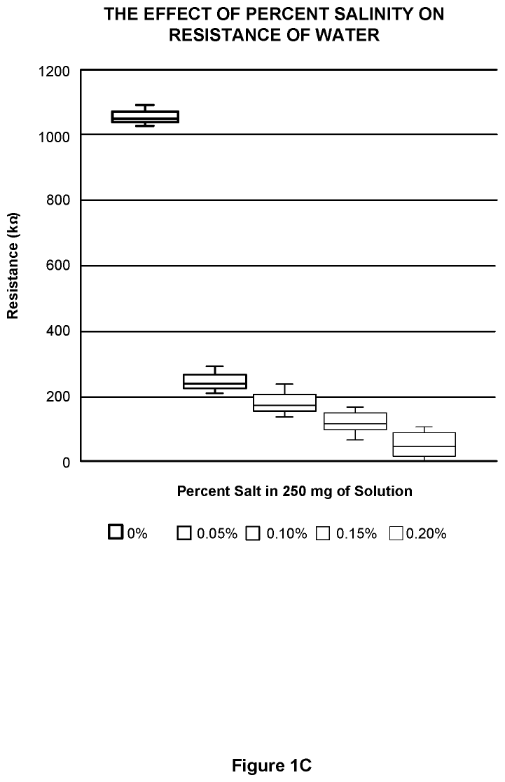

FIG. 1A is an overview of the components of a wearable device 100 for determining risk of an underlying health concern, according to an embodiment of the systems and methods described herein. In some embodiments, the components of wearable device 100 may include a power source 102, a resistance detector 104, a risk detector 106, storage component 108, wearable device casing 110, absorbing medium 112, warning signal generator 115, and a strap 114 for attaching the wearable device casing 110 to a user. In some embodiments, the components may be inside casing 110 and may be electrically connected to the absorbing medium 112 via wires 116 or other electrical conducting medium or structure. In some embodiments, the outer portion of casing 110 may include a display 118 for displaying visual user alters and/or a speaker 120 for presenting audible user alerts. Casing 110 may also transmit to the user haptic feedback to alert the user.

Power source 102 may comprise a battery (e.g., standard or rechargeable electrochemical battery) or other energy storage component capable of supplying power to the components of wearable device 100. In some embodiments, power source 102 may supply alternating current (AC) and/or direct current (DC) as needed by wearable device 110. Resistance detector 104 may comprise an ohmmeter that measures the electrical resistance of the absorbing medium and/or perspiration absorbed by the medium. Risk detector 106 may determine whether the detected resistance of perspiration meets at least one condition, and triggering appropriate alert signals if the at least one condition is met. Warning signal generator may generate appropriate warning signals to alert the user. Storage device 108 may comprise memory (e.g., RAM or ROM) for storing detected resistance values and programmable thresholds or conditions used by wearable device 100. In some embodiments, the internal components of wearable device 100 may be implemented as programmable logic on a circuit board or chip architecture. For example, the functions performed by resistance detector 104 and risk detector 106 may be performed by software or other code instructions that are performed by a programmable processor device or component.

FIG. 1B is an overview of the electrical “Ohm's Law” principles and programmable thresholds utilized by the wearable device for determining risk of an underlying health concern, according to an embodiment of the systems and methods described herein. For example, the basic circuit schematic shown may depict the electrical structure of a circuit formed by the absorbed medium 112 (which is represented by the resistance variable R1), power source 104 (which is represented by the voltage value 3.3V), resistance to ground (represented by R2 (1 MOhms), and the voltage V2 (which is the measurable voltage to ground over R2). The equations shown refer to the Ohm's Law principles (e.g., V=I*R) that are used by resistance detector 104 to determine the electrical resistance of the absorbed perspiration and/or absorption medium 112. The Ohms to salinity conversion chart depicts the range of electrical resistance to salinity and the underlying health conditions that may be present. For example, a measured perspiration resistance of 47 kohms (or less) may indicate the presence or risk of cystic fibrosis. Similarly, a measured perspiration resistance of 239 kohms (or higher) may indicate the presence or risk of hyponatremia. Measured perspiration resistance between 47 kohms and 239 kohms reflect a normal or health range.

FIG. 1C is a chart showing the relationship between electrical resistance (kohms) and salinity of a 250 mg solution, according to an embodiment of the systems and methods described herein. As shown, a solution having 0% salt would exhibit an electrical resistance of over 1000 kohms. A solution having 0.005% salinity exhibits just over 200 kohms resistance—0.10% salinity (just under 200 k ohms), 0.15% salinity (approximately 165 kohms), 0.20% salinity (approximately 75 kohms).

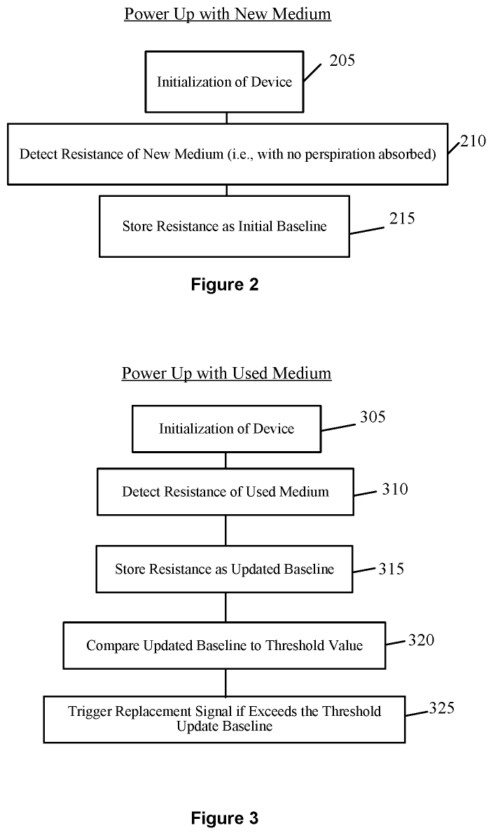

FIG. 2 is a method for powering up and detecting the electrical resistance of a new absorption medium of a wearable device for determining risk of an underlying health concern, according to an embodiment of the systems and methods described herein. At step 205, a wearable device 100 may be initialized (e.g., turned on). At step 210, resistance detector 104 may detect the resistance of a newly installed absorption medium (i.e., with no perspiration absorbed). At step 215, the detected resistance of the new medium may be stored in storage device 108 as an initial baseline resistance. The stored initial baseline resistance may then be used by wearable device 100 to determine the presence or risk of an underlying health concern as described herein.

FIG. 3 is a method for powering up and detecting the electrical resistance of a used absorption medium of a wearable device for determining risk of an underlying health concern, according to an embodiment of the systems and methods described herein. At step 305, a wearable device 100 may be initialized (e.g., turned on). At step 310, resistance detector 104 may detect the resistance of a previously used absorption medium (i.e., previously absorbed perspiration (which has dried, for example)). At step 315, the detected resistance of the used medium may be stored in storage device 108 as an updated or most recent baseline resistance. At step 320, the resistance detector 104 compares the updated baseline resistance to a threshold value. The threshold value may be stored in storage device 108 and be any resistance value associated with an unreliable or no longer useful absorption medium. At step 325, if the updated baseline resistance exceeds the threshold value, then resistance detector 104 may trigger a replacement signal indicating that the used medium should be replaced. In some embodiments, resistance detector 104 may set a new baseline at a given interval of time to account for saturation of the medium over time.

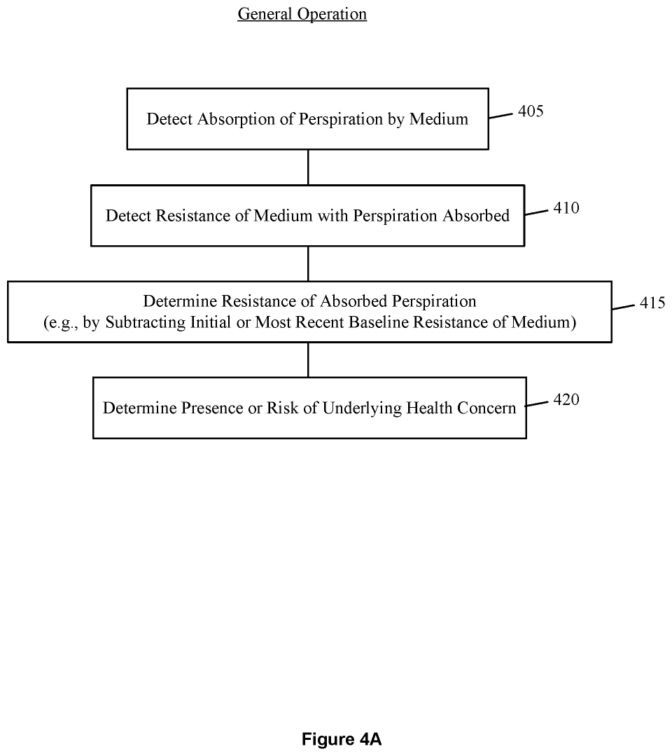

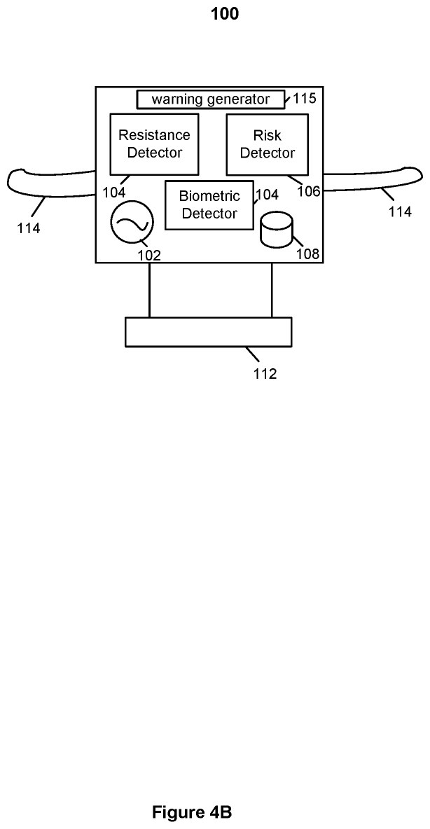

FIG. 4A is a method for determining risk of an underlying health concern, according to an embodiment of the systems and methods described herein. At step 405, resistance detector 104 may detect absorption of perspiration by the medium. At step 410, resistance detector 104 may detect the resistance of the medium with the perspiration absorbed. At step, 415, resistance detector 104 may determine the resistance of the absorbed perspiration. In some embodiments, the resistance of the absorbed perspiration may be determined by subtracting the initial or most recent baseline resistance of the medium (e.g., that is stored in storage element 108) from the total resistance of the medium and the absorbed perspiration. At step 420, the risk detector 106 may determine the presence or risk of an underlying health concern. In some embodiments, the presence or risk of an underlying health concern is determined by comparing the detected resistance of the absorbed perspiration to stored values corresponding to certain health concerns.

For example, if the detected resistance of the perspiration exceeds a predetermined value associated with a risk of hyponatremia (e.g., approximately (239 kOhms), then risk detector 106 may trigger a signal which causes warning generator 115 to alert the user (e.g., via video, audio or haptic feedback). Similarly, if the detected resistance drops below a predetermined value associated with a risk of cystic fibrosis (e.g., approximately 47 kOhms), then risk detector 106 may trigger a signal which causes warning generator 115 to accordingly alert the user (e.g., via video, audio or haptic feedback).

FIG. 4B is an overview of the components of a wearable device for determining risk of an underlying health concern, according to an embodiment of the systems and methods described herein. As shown, wearable device 100 (depicted in FIG. 1A) now includes at least one biometric detector 404 for detecting vital signs and other biometric signals of a user of wearable device 100. For example, a biometric detector may measure the user's heart rate, body temperature, respiration rate, and/or blood pressure. Based on data gathered by the biometric detector(s) 404, risk detector 106 may trigger alert signals if a condition are met. Example conditions include:

1. The user's heart rate is above 50%-85% of the user's maximum BPM (220−age) and the resistance of the medium and absorbed perspiration exceeds 239 ohms.

2. The user's body temperature is above 32 degrees Celsius, and the resistance of the medium and absorbed perspiration exceeds 239 ohms.

3. The user's respiration rate is above 40 breaths per minute, and the resistance of perspiration exceeds 239 ohms.

4. The user's systolic blood pressure is above 200 mmHg, and the resistance of perspiration exceeds 239 ohms.

5. Other conditions are possible.

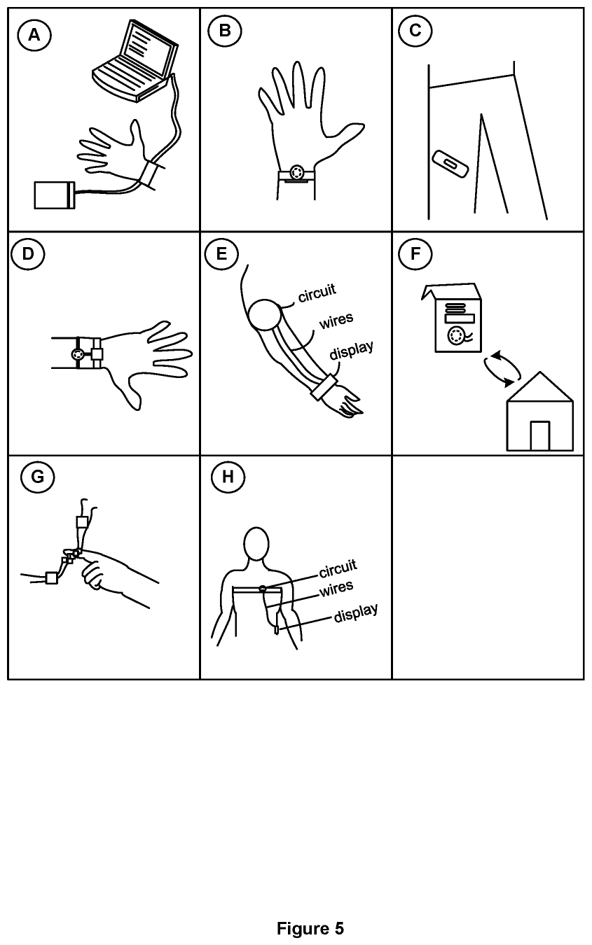

FIG. 5 is an overview of possible implementations of a wearable device for determining risk of an underlying health concern, according to various embodiments of the systems and methods described herein.

A—Depicts an absorbing medium connected to a power supply on one end and a computer on the other. The computer may run software that may perform some or all of the functions of the components of wearable device 100 described in FIGS. 1A and 4B.

B—Depicts a wristwatch-type device that may contain the various components of wearable device 100 described in FIGS. 1A and 4B.

C—Depicts a patch-type device that may contain some or all of the various components of wearable device 100 described in FIGS. 1A and 4B.

D—Depicts a variation of the wristwatch-type device described in B.

E—Depicts a device where a display is worn on the wrist and the absorbing medium and other components are positioned on the user's shoulder and connected to the display via wires.

F—Depicts a device that a wearable device that can communicate signals wirelessly to a home or other remote location.

G—Depicts a device where the medium is connected to the user's finger and the power supply and other components are connected to the medium via wires.

H—Depicts a device that straps to a user's chest.

Other embodiments, uses and advantages of the present invention will be apparent to those skilled in the art from consideration of the specification and practice of the invention disclosed herein. The specification and examples should be considered exemplary only. The intended scope of the invention is only limited by the claims appended hereto.

What is claimed is:

1. A wearable device for determining risk of an underlying health concern, comprising:
a replaceable medium for absorbing perspiration of a user, wherein the replaceable medium is at least occasionally in contact with the user;
a power source electrically connected to the medium;
a resistance detector processor for periodically detecting the electrical resistance of the absorbed perspiration, wherein the electrical resistance of the absorbed perspiration is dependent on the salinity of the perspiration;
a risk detector processor for: (1) determining whether the detected resistance meets at least one condition, and (2) triggering an alert signal if the at least one condition is met;
a storage device for storing detected resistance values; and
a warning signal generator for providing a warning signal to the user based on the alert signal.

2. The wearable device of claim 1 wherein the resistance detector processor comprises an ohmmeter.

3. The wearable device of claim 1 wherein the at least one condition is met if the detected resistance of the medium and perspiration exceeds a predetermined value associated with a risk of hyponatremia.

4. The wearable device of claim 3 wherein the predetermined value associated with a risk of hyponatremia is approximately 239 kOhms.

5. The wearable device of claim 1 wherein the at least one condition is met if the detected resistance drops below a predetermined value associated with a risk of cystic fibrosis.

6. The wearable device of claim 5 wherein the predetermined value associated with a risk of cystic fibrosis is approximately 47 kOhms.

7. The wearable device of claim 1 wherein the replaceable medium comprises an absorbent fabric.

8. The wearable device of claim 1 where in the replaceable medium comprises a gel.

9. The wearable device of claim 8 wherein the gel comprises a silica gel or activated carbon.

10. The wearable device of claim 1 further comprising a strap for securing the wearable device to the user.

11. The wearable device of claim 1 wherein the warning signal is an audible, visual, or haptic feedback.

12. The wearable device of claim 1 wherein the resistance detector further comprises a medium baseline resistance detector for: (1) detecting the electrical resistance of the medium with no perspiration absorbed; (2) comparing the detected electrical resistance of the medium with no perspiration absorbed to a baseline value stored in the storage device; and (3) generating a replacement signal if the detected resistance of the medium rises above a baseline value.

13. The wearable device of claim 12, wherein the medium baseline resistance detector further sets a new baseline at each interval of time that accounts for saturation of the medium over time.

14. The wearable device of claim 12 wherein the warning signal generator further provides a second warning signal to the user based on the replacement signal.

15. The wearable device of claim 13 wherein the warning signal is an audible, visual, or haptic feedback.

16. The wearable device of claim 1 further comprising at least one biometric detector for measuring the user's heart rate, body temperature, respiration rate, and/or blood pressure.

17. The wearable device of claim 15 wherein the at least one condition is met if the user's heart rate is above 50%-85% of the user's maximum BPM (220−age) and the resistance of the medium and absorbed perspiration exceeds 239 ohms.

18. The wearable device of claim 15 wherein the at least one condition is met if the user's body temperature is above 32 degrees Celsius, and the resistance of the medium and absorbed perspiration exceeds 239 ohms.

19. The wearable device of claim 15 wherein the at least one condition is met if the respiration rate is above 40 breaths per minute, and the resistance of perspiration exceeds 239 ohms.

20. The wearable device of claim 15 wherein the at least one condition is met if the systolic blood pressure is above 200 mmHg, and the resistance of perspiration exceeds 239 ohms.
